(19) United States
(12) Patent Application Publication
(10) Pub. No.: US 2008/0268287 A1
(43) Pub. Date: Oct. 30, 2008

(54) Method of Converting Water-Soluble Active Proteins Into Hydrophobic Active Proteins, the Use of the Same for the Preparation of Monomolecular Layers of Oriented Active Proteins, and Devices Comprising the Same

(76) Inventors: Elena Donadio, Pisa (IT); Ettore Balestreri, Pisa (IT); Romano Felicioli, Crespina (IT)

Correspondence Address: Beusse Wolter Sanks Mora & Maire, 390 N. ORANGE AVENUE, SUITE 2500, ORLANDO, FL, 32801, US

(21) Appl. No.: 12/093,031

(22) PCT Filed: Jun. 30, 2006
(86) PCT No.: PCT/IB06/52203

§ 371 Date: Jun. 17, 2008

(30) Foreign Application Priority Data
Nov. 11, 2005 (EP) PCT/EP2005/055901

(51) Int. Cl. B32B 9/04; C07K 14/00; C12N 9/76; C12N 9/26; C12N 9/24; C12N 9/20; C12N 9/48; C12N 9/66; C12M 1/16; G01N 33/566

(52) U.S. Cl. 428/704; 530/402; 435/213; 435/201; 435/200; 435/198; 435/212; 435/218; 435/289.1; 436/501

(57) ABSTRACT

The present invention relates to a method of converting hydrophilic active proteins (HPiAP) into hydrophobic active proteins (HPoAP) suitable for the anchorage in their active form on hydrophobic substrates. The present invention also relates to the preparation of ordered monomolecular layers of oriented active proteins immobilized onto hydrophobic solid supports to be used for mechanical manipulation and investigations, including Atomic Force Microscopy (AFM) in aqueous solutions and assays employing the same devices.

### TECHNICAL FIELD OF THE INVENTION

The present invention relates to a method of converting hydrophilic, water-soluble, active proteins (HPiAP) into hydrophobic active proteins (HPOAP) by covalent modifications of hydrophilic amino-acid surface residues with hydrophobic reactants in heterogeneous phase or in differential-polarity solvent mixture. The hydrophobized proteins spontaneously self assemble onto many different hydrophobic solid supports in statistically oriented monolayers, suitable for the realization of bioreactors and biosensors, and for mechanical manipulation and inspections including Atomic Force Microscopy (AFM).

### BACKGROUND OF THE INVENTION

A large variety of experimental bioengineering investigations and/or applications demand monolayers of biomolecules firmly anchored to a solid substrate. The case of Atomic Force Microscopy (AFM) is the most evident instance. The Atomic Force Microscope allows scanning of samples fixed on solid supports at the level of atomic dimensions. The signals sensed by the flexible cantilever, after the adequate amplification, allow the topographical analysis of the examined sample. Therefore, Atomic Force Microscopy (AFM) allows the examination of samples with nanoscopic dimensions, i.e. between 1 and 200 nm. When the scanning is performed in an aqueous solution it is also possible to analyze biological samples under physiological conditions. However, to obtain images corresponding to reality it is mandatory that the examined biological sample be steadily fixed on a support, so that the tip of the microscope cannot move it while performing the scanning. Furthermore, the sample should preferably be organized as a monomolecular layer.

The methods presently known to prepare monomolecular layers immobilized onto a hydrophobic substrate, exploiting hydrophobic interactions, imply either the use of naturally hydrophobic molecules, see Dawn S. Y. Yeo et al. “Combinatorial Chemistry & High Throughput Screening” 2004, Vol. 7, No, 3 pp. 213-221, or the use of water soluble proteins acquiring hydrophobic properties from conformational changes due to temperature, ionic strength or pH modification, see Kapila Wadu-Mesthrige et al. “Journal Scanning Microscopy”, 2000, Vol 22, pp. 338-388; Hyun J. et al. “J. Am. Chem. Soc” 2004, 126(23) pp. 7330-7335. These methods lack selectivity since the chemical-physical modifications may also involve the active site of the protein, with consequent loss of the native biological activity.

Alternatively, a hydrophobized protein may be prepared through processes of genetic engineering of the water-soluble proteins, which however are very complex and do not guarantee the conservation of the original activity, see Hyun J. et al. (above) or C. Tranchant et al. “Rev. Neurol. (Paris)” 1996, 152 (3), pp. 153-157.

For these reasons, up to now it has been possible to successfully examine only membrane proteins, which are spontaneously fixed to a lipid double layer, or molecular complexes having high dimensions. No images of soluble proteins have been reported.

Scope of the present invention is therefore that of providing novel methods for hydrophobizing water-soluble hydrophilic functional proteins without affecting the native biological function of the protein, and means enabling scanning, imaging and manipulating water-soluble proteins.

### SUMMARY OF THE INVENTION

The present invention relates to methods for converting hydrophilic, water-soluble, functional proteins (HPiAP) into hydrophobic proteins (HPOAP), while maintaining their original biological activity. The methods imply the asymmetrical chemical modifications of the hydrophilic proteins, that is that they introduce chemical modifications on the amino-acid surface residues situated in a part of the molecule distal from, or opposite to, the part responsible for the protein function. This result is achieved by carrying out the reaction with the “hydrophobizing” reactants in heterogeneous phase or in a homogeneous differential-polarity solvent mixture.

The present invention is based on the knowledge that water-solubility of proteins is due to the balance of the number of hydrophobic and hydrophilic amino-acid residues occurring on their surface. In general, in active proteins such as enzymes, a variously extended area of the surface is deputed to interact with small water-soluble molecules of different nature, namely substrates, inhibitors, cofactors and others, and this area, i.e. the active site, is mainly hydrophilic.

The method of the invention exploits the asymmetrical distribution of polarity to direct the chemical modification reaction necessary to change the nature of the protein in the area spaced from or opposite to this active site.

The so obtained “hydrophobized” functional proteins is then immobilized or anchored onto hydrophobic solid supports through hydrophobic interactions in order to obtain oriented monomolecular layers of active protein.

The term “oriented” used in the present invention means that the immobilized proteins are essentially all oriented with the hydrophobized part of the molecule attached to the surface of the substrate, while the free active site being distal from the point of attachment. The term does not mean that the immobilized molecules show all the same spatial orientation, which on the contrary is random.

The term “monomolecular layer” means that the obtained layers are mostly and preferably “monomolecular”, although the formation of undesired limited areas of multimolecular layers may not always be avoided.

Accordingly, a first object of the invention is a method of preparing a hydrophobic or partially hydrophobic active protein as disclosed in anyone of claims 1 to 8.

A second object of the invention is a naturally hydrophilic active protein made hydrophobic active protein as disclosed in claim 9 or 10.

A third object of the invention is a method of preparing a device as disclosed in claims 11 and 13 and the so obtained device.

Further objects of the invention are bioreactors or biosensors comprising the device.

Still further objects of the invention are assays to investigate the conformation of a water-soluble protein or the conformation of its complex with a ligand or the interaction between the protein and a ligand or ligand-candidate thereof as disclosed in claims 17 and 18.

Final object of the invention is the use of the bioreactor or biosensor as solid reactive support for the preparation of micro arrays for diagnostic, genetic, immunological analysis or for mechanical manipulation of protein or genetic material, or for the treatment of liquids or gazes.

There are many factors normally capable of influencing the functions of an active protein as an enzyme. For instance conformational effects when conformational or charge-distribution modifications occurs; steric effects, when the interaction of the substrate with the enzyme is affected by steric hindrance; partitioning effects, related to the chemical nature of the support material and to the modified microenvironment.

Therefore, it was a priori expectable that a hydrophobization reaction could change the kinetics and other properties of the active protein, for instance affecting the enzyme-specific activity.

On the contrary, the results reported in the examples show that the claimed method eliminate or minimizes this expectable loss of activity.

### BRIEF DESCRIPTION OF THE FIGURES

FIG. 1. The figure illustrates the development from t0h to t6h of BAPNA hydrolysis caused by cholesteryl-trypsin immobilized onto silicon slab.

FIG. 2. The figure reports the topography of cholesteryl-trypsin hydrophobically anchored in water solution to a silicon slab (150 sq nm area scanning). Panel (a) shows the image of a 1 μm area; panel (b) shows the image of 150 nm area; panel (C) represents the cross-section of the monomolecular layer.

FIG. 3. The figure reports the topography of cholesteryl-trypsin anchored in water solution to silica slab (50 sq nm area scanning). Panel (a) shows the image of the 50 nm area; panel (b) shows the cross-section of the monomolecular layer.

FIG. 4. The figure reports the topography of cholesteryl-trypsin anchored in water solution to silica slab (24 sq nm area scanning). Panel (a) shows the image of the 24 nm area; panel (b) shows the cross-section of the monomolecular layer.

FIG. 5. The figure reports the mass-spectra of distilled water (panel above) passed by the functionalized tryptic tip device: the trypsin autolysis peaks are absent; and trypsin in solution (panel below). The autolysis peaks are clearly evident.

FIG. 6. The figure illustrates the mass-spectra of Human Serum Albumin (HSA) (panel above) digested for 10 min by the functionalized tryptic tip device: the trypsin autolysis peaks are absent, while the HSA peaks are fully present. (Panel below) HSA digested overnight by trypsin in solution: the circle shows an autolysis peak.

FIG. 7. The figure shows the kinetics of quartz dumping induced by blocking molecules of trypsin modified according to the strategy II of the invention. The quartz surface is covered by a very tiny gold lamina, made hydrophobic by thiocholesterols.

### DETAILED DESCRIPTION OF THE INVENTION

The hydrophilic active proteins (HPiAP) according to the present invention are any water-soluble protein selected from the group comprising enzymes, hormones, receptors, antigens, antibodies, allergens, immunoreactive proteins, affinity partners and any derivatives, fragments, complexes functionally equivalents thereof. In particular, the active protein may be any enzyme in its native form selected from the general classes of oxidoreductases, transferases, hydrolases, lyases, isomerases and ligases. That is anyone of the enzymes actually in use in the basic and applied research and industrial application, such as trypsin, muscle aldolase, amylase, lipase, collagenase or elastase.

Usually in the practice of chemical modification of proteins both the protein and the reactants are in water solution (homogeneous reaction). However, two main restrictions characterize the instant invention: the modified protein, for instance an enzyme, must retain its activity and the reactants are little or completely insoluble in water.

Therefore, in order to convert the hydrophilic active protein into a hydrophobic or partially hydrophobic protein, while maintaining its functionality, two different strategies have been developed, both aimed at “hydrophobizing” parts of the protein distal from, or opposite to the active site, which must be preserved in its native conformation and nature.

[Strategy I] A first strategy implies a heterogeneous solid/liquid system. The active site, and the surrounding area, is reversibly protected by attaching it to a solid matrix by affinity chromatography. The solid phase bears on its surface a ligand specific for the active site of the functional protein. The “hydrophibizing” step is then carried out on the immobilized protein. The reactant is dissolved in a buffered water solution and used as the eluant, i.e. the liquid phase. In this way, the protein molecule is forced to expose to the eluant only that part far from the active site.

Any commercially available affinity matrix suitable for chromatography may be used according to the invention, for instance dextrane-based or cellulose-based resins, such as phosphocellulose. Known materials are fitted with functional groups suitable for linking any type of ligand such as substrate analogues, reversible inhibitors, cofactors. Therefore the preparation of the affinity solid phase and the conditions for loading the hydrophilic protein and subsequently eluting the hydrophobized protein are well known to the person skilled in the art. The elution is normally carried out by affinity elution.

According to this method the molecules accidentally modified in the active site amino-acid residues result separable in the elution step.

[Strategy II] A second strategy implies a liquid/liquid mixture obtained by using the hydrophilic protein dissolved in a polar solvent (usually water), and the hydrophobic reactant dissolved in a less-polar solvent, at least partially miscible with water. According to the differences in polarity between the solvents, and on the volumes involved, two reaction regimes are possible:

a) the two solvents are partially immiscible: two macroscopic phases are generated, and the reaction between the hydrophilic protein and the hydrophobic reactant takes place at the interface between the two phases. In this situation, the protein tends to orient its active site towards the polar phase, since the active site and its surroundings is the most polar region of the protein surface. The residues involved in the hydrophobization reaction are, therefore, located far from the active site, thus preserving protein activity. This embodiment of the invention is less preferred because the protein tends to precipitate at the interface, with resulting decrease in final yield.

b) in the preferred regime, the two solvents are miscible. In this case, different salvation microenvironments are created in the mixture at molecular level. The hydrophobic moiety of the reactant molecules is insoluble in the polar solvent, then, in the mixture, it is solvated by less-polar solvent molecules. On the other hand, polar solvent molecules are surrounding the more hydrophilic side of the protein molecules. The hydrophobization reaction follows a polarity gradient: the most polar region of the hydrophobic reactant tends to react with the less polar region of the protein (i.e. the region far from the active site). Even in this case, the probability of an involvement of residues belonging to the active site region is very low, and therefore the activity is preserved.

To further ensure the activity preservation, optionally one can protect the amino-acids responsible for the activity or engaged in the recognition, interaction, and catalytic processes is improved, by co-dissolving in the same aqueous phase protecting molecules such as substrates, substrate-analogues and/or competitive inhibitors.

After the reaction the chemically modified protein may be purified by hydrophobic interaction chromatography, or by dialysis, or by other purification methods.

Polar solvent, or aqueous solution, means any solution in water, neutral for the protein function.

For less-polar solvent is meant any well known solvent partially soluble or insoluble in water, for example alkyl alcohols, alkyl amines, halogenated alkanes, etc.

Chemical Modification

Many amino acids residues have functional groups in the side-chain such as —NH₂, —OH, —SH and therefore are suitable for reaction with the hydrophobizing reagent: they are Thr, Met, Trp, Lys, Arg, Asp, Cys, Glu, H is, Ser, Tyr. From this list, the amino acids having side-chain amide groups and hydrocarbon side-chains and glycin have been excluded because of their relatively low concentration on the exposed protein surfaces and, even more important, because of their non-reactive hydrophobic side-chain. A large number of possible modifications is therefore predictable.

Any chemical modification caused by chemical reagents capable of either converting one or more hydrophilic amino-acid residue(s) into hydrophobic residues or capable of attaching a large highly hydrophobic moiety to a suitable polar amino-acid may be used to convert the hydrophilic water-soluble protein into a hydrophobic one. This conversion may occur either on identical or different amino acid residues.

Suitable hydrophobizing reagents are chemical compounds capable of performing any covalent modification including but not limited to O—, N—, S-alkylation, O—, N—, S-acylation, diazo-linkage, peptide bond formation, arylation, Schiff's base formation, and the like. Examples of these reagents are acylanhydrides, acylchloride, diazocompounds, aldehydes, ketones, but also compounds capable of integrating large lipophylic moieties such as cholesteryl-chlorophormate or equivalents.

The resulting modified protein is amphipatic with higher hydrophoby than the native form, is characterized by asymmetrical distribution of the non-polar/polar residues and maintains its water-solubility and its original functionality since the active site was protected in the course of the reaction.

According to both strategies the method may comprise an optional step of separation of the hydrophobized active proteins from the accidentally inactivated protein. Any protein purification procedure, which exploits either the affinity of the protein for a specific substrate or the greater hydrophobicity of the modified proteins, can be used to purify the active product. They include affinity chromatography and affinity elution procedures or water washing of the protein molecules directly on the hydrophobic AFM-substrate.

There are many factors normally capable of influencing the functions of an active protein as an enzyme. For instance conformational effects—when conformational or charge-distribution modifications occurs; Steric effects—when the interaction of the substrate with the enzyme is affected by steric hindrance; partitioning effects, related to the chemical nature of the support material and to the modified microenvironment.

Therefore, it was expectable that the hydrophobization according to the present invention could change the kinetics and other properties of the active protein, for instance a decrease of the enzyme-specific activity.

On the contrary, the results reported hereinafter in the examples show that the claimed method eliminate or minimizes the expectable loss of activity.

Immobilization

The hydrophobized active proteins obtained according to the present invention are suitable to be bound to a hydrophobic substrate through hydrophobic interactions. These latter result in assembling an ordered layer, preferably monomolecular layer of protein onto the surface of the substrate. Moreover, the hydrophobic forces turn on to be strong enough to allow the water washing of the unmodified molecules, thus availing highly efficient purification means.

Suitable hydrophobic substrates are any support, naturally hydrophobic or made hydrophobic by derivatization. For instance the substrate may be rendered hydrophobic by coating it with functionalized lipids such as thiolated lipids, for example thiocholesterols or other equivalent lipids capable of reacting with the surface of the substrate. The substrates include, but are not limited to, natural polymer such as cellulose or synthetic polymers, such as PVC, poly(meta)acrylates, nylon, polyethylene, teflon, carbon materials, silicon, glass, resins, metal particles or metal sheets, paper and the like. The support can be in the form of wafers, slabs, laminas, sheets, tubes, fibers, particles, granulates, powders all in macro, micro, or nano-size, and may either have atomic flat, smooth surface or rough surface.

The complex made by the hydrophobized active proteins immobilized onto the hydrophobic or hydrophobized substrate is a device suitable for preparing bioreactors o biosensors. These devices are in form of “activated” wafers, slabs, laminas, sheets, tubes, particles, fibers, granulates, powder, all of macro, micro, or nano-size.

The devices of the invention are assembled in bioreactors or biosensors. For instance granulates of activated particles may be used to built-up cartridges packed in a common laboratory tip or to built-up filters packed in a case or in a column. Slab, lamina and sheets may be assembled in kits for micro arrays for diagnostic, genetic, immunological analysis or for mechanical manipulation of protein or genetic material.

Our results show that the derivatized and anchored molecules do not significantly differ, both structurally and functionally, from the native ones. The examples show, for instance, that the enzymes maintain most of their native activity.

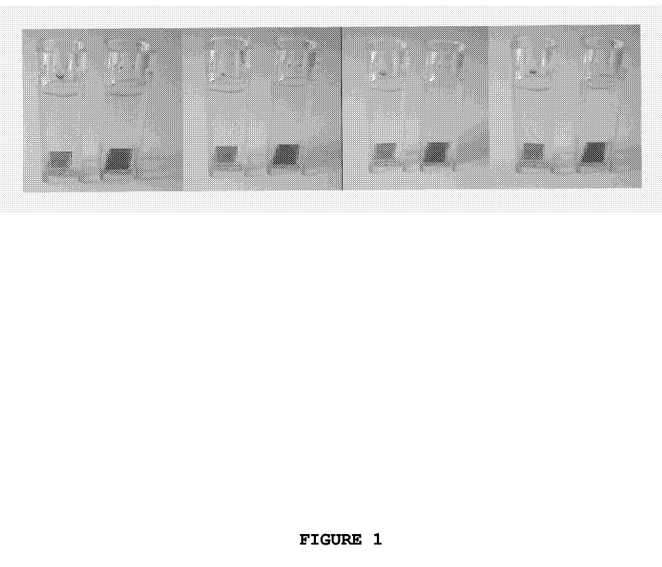

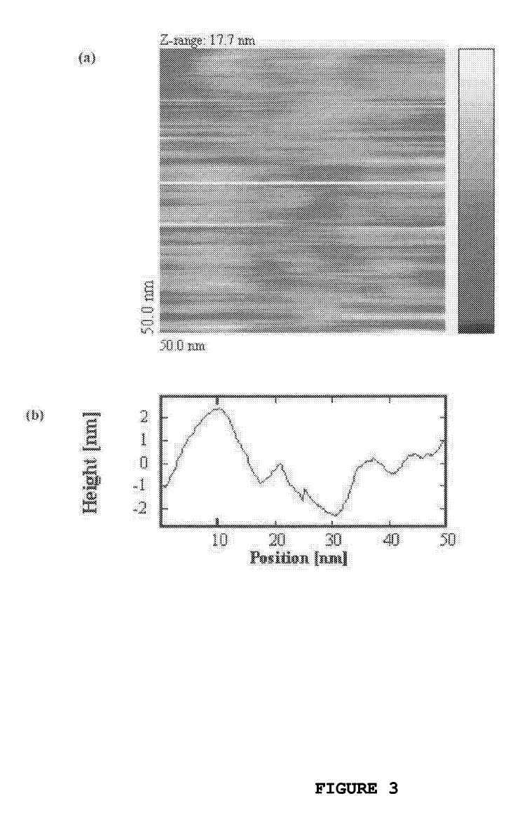

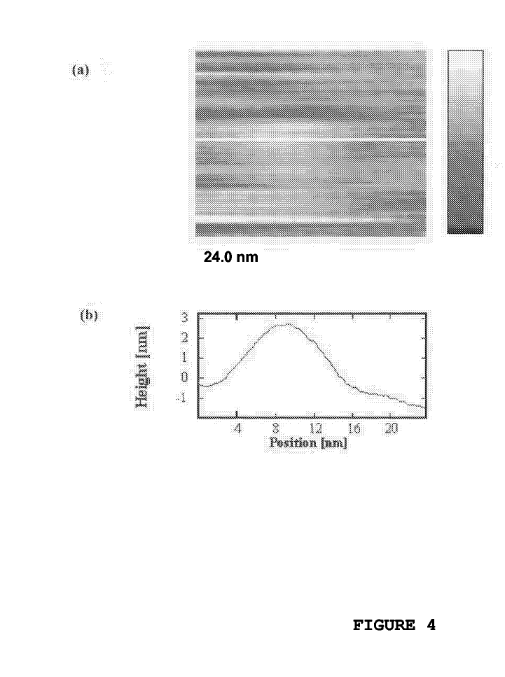

AFM observations and enzymatic activity assays of enzyme coated silica slabs show that the active conformation is retained in the adhered molecules, whereas the homogeneous appearance at AFM scanning along with the calculated dimensions of the modified protein indicate that the inactive or wrongly modified molecules have been washed away (see FIGS. 1, 2, 3 and 4).

Even more importantly is the fact that the hydrophobic interactions are so strong and stable to allow a repeated use of the protein-coated support in a water medium, for instance repeated enzymatic assays.

As the molecules anchored on the hydrophobic support are chemically modified in a region that does not contain the active site, they are, in the aqueous medium, all oriented with the active site facing the water phase. This is a favorable condition to investigate at atomic resolution the interaction mechanism of the active protein with several small molecules such as substrates, substrate analogues, allosteric effectors, inhibitors, antibody etc.

Moreover, since each protein molecule is stably anchored to the support, this fixed conformation prevents occasional contacts between molecules, so preventing undesirable damaging reactions. Thus, accidental contacts with molecules (i.e. substrates, inhibitors, effectors etc.) occurring in the water solution are under the experimental control. These conditions render the ordered monomolecular layer of oriented active proteins enduring bioreactors or biosensors, suitable to be used in medicine, industrial and environment analytical chemistry. The instant invention provides an easy procedure for making such biosensors using the several hundreds of water-soluble enzymes or other active proteins currently in use in any field of applied biology.

The invention also relates to assays making use of the claimed bioreactors and biosensors to investigate the conformation of a water-soluble active protein or the conformation of its complex with a ligand by imaging the monomolecular layer of immobilized oriented protein and/or its complexes by atomic force microscopy (AFM). This type of assay is particularly useful to investigate the interaction between a water-soluble protein and ligand candidates in order to identify new ligands. The immobilized proteins of the invention are also suitable as solid reactive support for the preparation of micro arrays for diagnostic, genetic, immunological analysis or for mechanical manipulation of protein or genetic material.

Finally the derivatization protocol can be modified in order to obtain more extensively “hydrophobized” molecules or molecules randomly modified in different surface regions, including the active site. A large number of modified molecules are therefore obtainable. In aqueous medium, all of them will spontaneously arrange with a random orientation to form a layer on the hydrophobic substrate. The contemporary use of the active form image and of the multiple differently oriented images of the same molecule will allow the computerized accurate reconstruction of the image of the original molecule under investigation.

The invention is further described in details in the following examples which are simply intended to describe specific embodiments of the invention, without limiting the scope of protection.

Example 1 and 2 describe the two general strategies envisaged by the invention for preparing the claimed protein-coated support for bioengineering applications in water solution.

Two water-soluble native enzymes, namely muscle Aldolase and Trypsin were covalently modified in vitro by alkylating primary amino-groups to obtain amphipatic molecules.

### EXAMPLE 1

(Strategy I): The used HPiAP was rabbit muscle Aldolase and the alkylating agent was acetic anhydride. The derivatization was carried out in heterogeneous phase. The HPiAP was absorbed per affinity chromatography on a phosphocellulose (analogous of the substrate fructose-1, 6-bisphosphate) column, reacted with the percolating aqueous solution of the acetic anhydride and then eluted with the substrate fructose-1,6-bisphosphate. Titration of Lysyl residues indicated that 15 residues per molecule have been modified. The recovered HPOAP resulted active with unvaried kinetic parameters. The aldolase activity was assayed according to Racker using fructose-1,5-bisphosphate as substrate and glyceraldehyde phosphate dehydrogenase and triosophosphate isomerase as ancillary enzymes (see Table I).

TABLE I

| Kinetic | | 15 acetylated |
|---|---|---|
| parameter | native | Lysyl residues |
| Vmax | 2000 | 3200 |
| Km (μM) | 23 | 22 |

### EXAMPLE 2

(Strategy II): The HPiAP was trypsin and the alkylating agent was cholesteryl chlorophormate in propanol solution. The water solution of trypsin was mixed with the propanol solution of cholesteryl chlorophormate under vigorous stirring for 30 minutes. After termination of the reaction the hydrophobized trypsin was collected.

After the reaction the chemically modified protein is purified by hydrophobic interaction chromatography using Phenyl HS resin, and eluted with a 0-30 mM ammonium sulfate in 20 mM Gly-HCl buffer. The fraction constituting the main peak of both protein and activity were used for the immobilization experiments. After derivatization, the trypsin activity was assayed at pH8 using Nα-benzoyl-DL-arginine-p-nitroanilide (BAPNA) as synthetic substrate. HPOAP shows full activity, unvaried kinetic parameters and 3 lysyl residues modified per molecule (see Table II).

A silica gel was dipped in the solution of cholesteryl-Trypsin, repeatedly washed with water, immersed in the solution of the synthetic substrate BAPNA buffered at pH 8 and incubated at 37° C. for the reported time. A silicon slab immersed in the same solution was used as a control. The appearance of the yellow color indicates that the anchored cholesteryl-trypsin retained proteolytic activity after several water washings (see FIG. 1).

The immobilized enzyme was active for months as assayed by spectrophotometric methods using BAPNA (Nα-benzoyl-DL-arginine-p-nitroanilide) as the substrate indicating a strongly enduring hydrophobic binding between the amphipatic enzyme molecule and the hydrophobic silica slab.

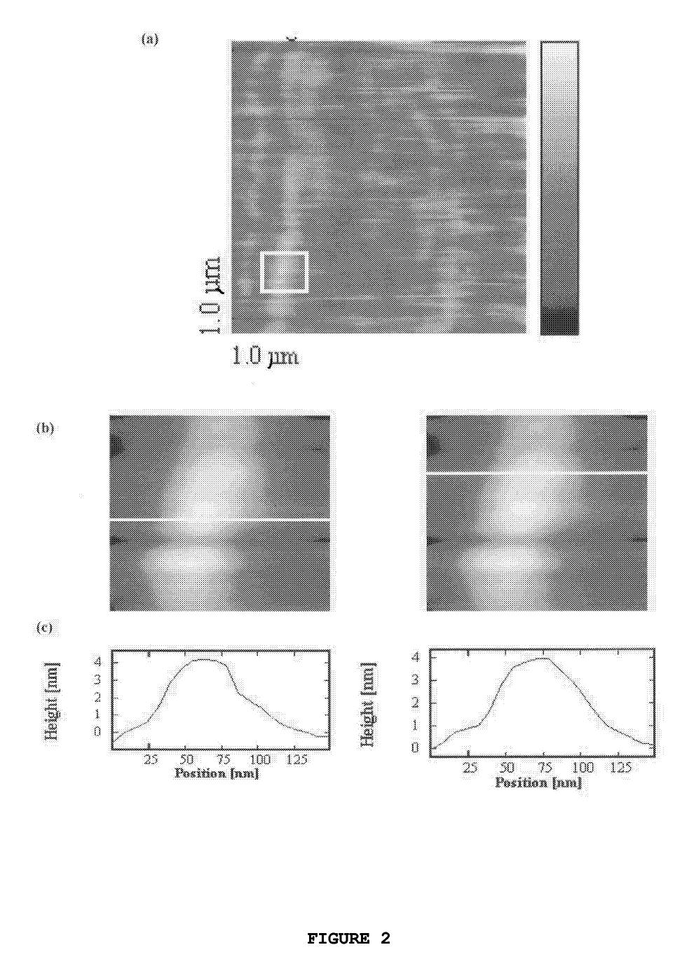

The silica slab coated with cholesteryl-Trypsin was scanned by AFM at 10⁻⁸ N and 1000 nm/sec. The imaging is reported in FIGS. 2, 3 and 4

TABLE II

| Kinetic | | 3 cholesteryl |
|---|---|---|
| parameter | native | lysyl residues |
| Specific activity | 1, 1 | 0, 90 |
| Km (μM) | 1, 67 | 1, 68 |

### EXAMPLE 3

The enzyme Adenosine Deaminase was hydrophobized according to the liquid/liquid strategy II of the present invention. The hydrophobizing agent was represented by cholesteryl chlorophormate dissolved in propanol solution.

### EXAMPLE 4

The enzyme Citosine Deaminase was hydrophobized according the liquid/liquid strategy II of the present invention. The reaction scheme was similar to that of example 2.

### EXAMPLE 5

The Enzyme Glutamic-Oxaloacetic Transaminase was hydrophobized according the liquid/solid strategy I of the present invention using Pyridoxal phosphate-bound resins as the affinity chromatography solid phase.

### EXAMPLE 6

The Enzyme Alanine-Pyruvate Transaminase was hydrophobized according the liquid/solid strategy I of the present invention using Pyridoxal phosphate-bound resins as the affinity chromatography solid phase.

### EXAMPLE 7

The enzyme Malic Dehydrogenase was hydrophobized according the liquid/solid strategy I of the present invention using Nicotinamide-bound resins as the affinity chromatography solid phase.

### EXAMPLE 8

The enzyme Alcohol Dehydrogenase was hydrophobized according the liquid/solid strategy I of the present invention using Nicotinamide-bound resins as the affinity chromatography solid phase.

### EXAMPLE 9

The enzyme Lipase was hydrophobized according the liquid/solid strategy I of the present invention using Propionylacetate-bound resins as the affinity chromatography solid phase.

### APPLICATION EXAMPLES

### EXAMPLE 10

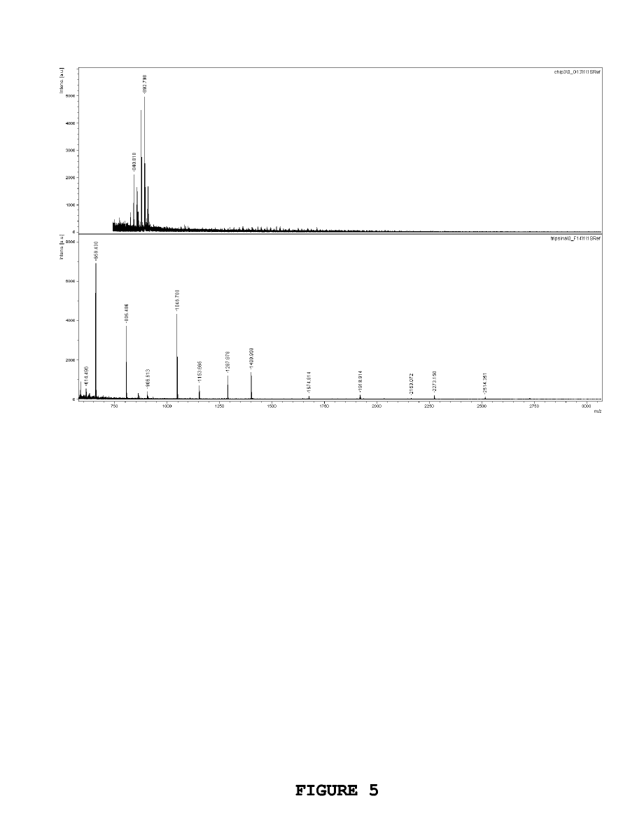

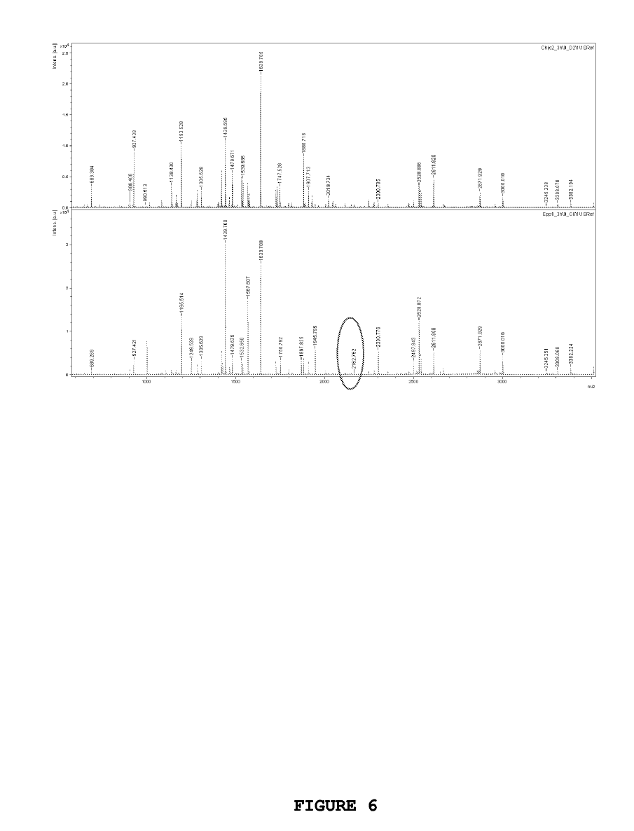

A bioreactor, useful to digest proteins in solution, is realized by derivatizing bovine trypsin like in the Example 2, and blocking it on PVC (poly-vinyl-chloride) granules. These granules are used to built-up a cartridge packed in a common laboratory tip. This device is able to digest protein in solution with a strong enhancement of the digestion reaction performances. In particular, compared with the classic protocol of protein digestion by a direct addition of trypsin in the solution, three are the main improvements obtained by using the functionalized tip device:
a) this device does not release trypsin molecules in the solution;
b) this device reduces (or even eliminates) trypsin autolysis fragments; (see FIG. 5 and FIG. 6)
c) the digestion time required for a good protein analysis via mass spectrometry is reduced from the overnight period of the classic in solution digestion protocol to only a few minutes (from 1 up to 10 minutes, depending on the concentration of the protein to be digested) (see FIG. 6).

### EXAMPLE 11

A bioreactor is build, analogous to that described in Example 10, in which several cartridges of PVC powder binding different hydrophobized hydrolytic enzymes are joined together in a pipeline. This device is useful to purify waste waters of industrial activities. The specific enzymes used in the cartridges changes depending on the composition of the waste water to be purified: for instance, to purify a bakery waste water, cartridges activated with amylase and trypsin are used. To purify waste water from tannery, cartridges activated with lipase, collagenase and elastase are used.

### EXAMPLE 12

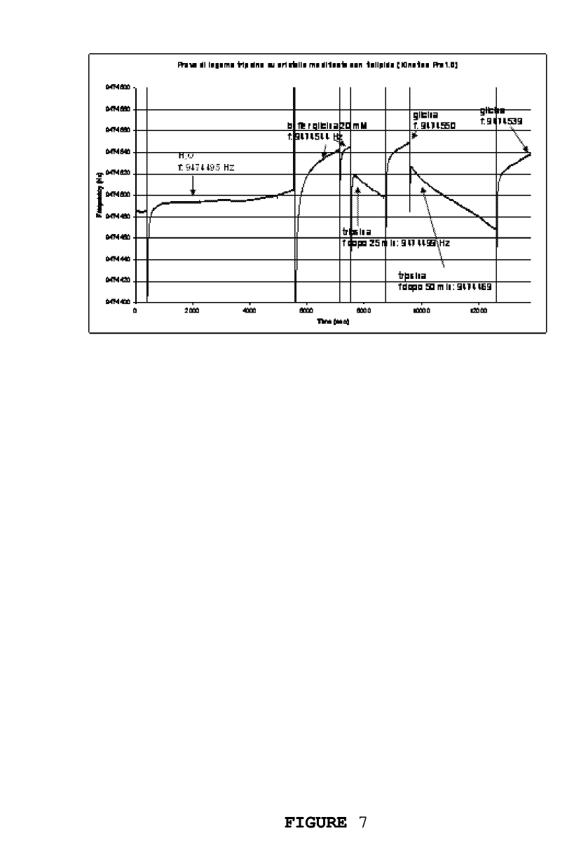

A biosensor is realized where a quartz disk is covered by a tiny gold surface. This surface has been hydrophobized by covering it with a layer of thiocholesterols, that react spontaneously with the gold, producing an ordered layer. This hydrophobized surface is suitable for blocking proteins hydrophobized obtained by both the proposed strategies. The protein blockage can be measured by evaluating the amount of the dumping induced by protein mass on quartz spontaneous oscillation frequency (see an example in FIG. 7). The active protein blocked can be used as probes for any specific protein-protein interaction. The link between the probe and a specific factor to recognize can be read as a further dumping in the quartz oscillation.

1. A method for preparing a hydrophobic or partially hydrophobic active protein comprising the steps of reacting an active water-soluble protein with a reagent capable of converting one or more hydrophilic amino acid residues into hydrophobic residues, wherein the active site responsible for the protein activity is protected by operating the hydrophobization reaction in homogeneous differential-polarity solvent mixture.

2. The method according to claims 1, comprising the steps of: dissolving the active water-soluble protein in an aqueous solution; dissolving the reagent in an less-polar solvent, at least partially miscible with water; preparing a polar/less-polar homogeneous mixture; whereby reacting the protein and the reagent; recovering the hydrophobized active protein.

3. The method according to claim 2, further comprising dissolving in the aqueous solution a reversible ligand specific for the active site of the protein.

4. The method according to claim 1, wherein the reagent capable of transforming hydrophilic amino acid residues into hydrophobic residues is selected from the group comprising alkylating, acylating, arylating, diazo, peptide-bond forming, Schiff's base forming compounds, or compounds capable of introducing a hydrophobic moiety.

5. The method according to claim 1, wherein the active water-soluble protein is selected from the group comprising enzymes, hormones, receptors, antigens, antibodies, allergens, immune-reactive proteins, affinity partners and derivatives, fragments and functionally equivalents thereof.

6. The method according to claim 5, wherein the active water-soluble protein is selected from the group comprising muscle aldolase, trypsin, amylase, lipase, collagenase or elastase.

7. A hydrophobized active protein obtainable by the method according to claim 1, having in a part of the protein distal from, or opposite to, the region responsible for the activity one or more hydrophilic aminoacid residues converted into hydrophobic residues.

8. The hydrophobized active protein of claim 7, having one or more hydrophilic aminoacid residues converted into cholesteryl-aminoacid residues.

9. A method of preparing a device consisting of a monomolecular layer of oriented active protein immobilized onto a solid support comprising the step of contacting the hydrophobized protein of claim 7 with a hydrophobic solid support.

10. The method according to claim 9, wherein the hydrophobic solid support is natural or synthetic polymers, carbon materials, silicon, metal, resins, all optionally derivatized to make them hydrophobic.

11. The method according to claim 10 wherein the hydrophobic solid support is in the form of wafers, slabs, laminas, sheets, tubes, fibers, particles, granulates, powders.

12. A device obtainable by the method according to claim 9.

13. The device according to claim 12 in the form of a cartridge packed in a laboratory tip or in the form of a filter or in the form of a microarray support.

14. A bioreactor or a biosensor comprising, or consisting of, the device according to claim 13.

15. A technology to investigate the interaction between a water-soluble protein and a ligand or ligand candidate comprising the step of:
preparing a device according to claim 13;
contacting the immobilized protein with the ligand or ligand candidate and
imaging the protein and/or its complex by atomic force microscopy (AFM).

16. A technology to investigate the conformation of a water-soluble protein material comprising the step of:
converting the protein material into a randomly hydrophobized material;
contacting the hydrophobized material with a hydrophobic solid support to obtain a monomolecular layer of randomly oriented immobilized protein material;
imaging the material by atomic force microscopy (AFM);
reconstructing the image of the Original material by computer assisted means.

17. The use of the device according to claim 13 for the preparation of micro arrays for diagnostic, genetic, immunological analysis or for mechanical manipulation of protein or genetic material.

18. The use of the device according to claim 13 for the preparation of bioreactors for the treatment of liquids or gazes.

19. A method of preparing a device consisting of a monomolecular layer of oriented active protein immobilized onto a solid support comprising the first step of preparing a hydrophobic or partially hydrophobic active protein by reacting an active water-soluble protein with a reagent capable of converting one or more hydrophilic amino acid residues into hydrophobic residues, wherein the active site responsible for the protein activity is protected by operating the hydrophobization reaction in heterogeneous phase, and a second step of contacting the so obtained hydrophobized protein with a hydrophobic solid support.

20. The method of claim 19, wherein the hydrophobization reaction comprises the steps of: immobilizing the active water-soluble protein through its active site onto an affinity matrix; reacting the immobilized protein with the reagent; eluting the hydrophobized active protein from the matrix.

21. The method according to claim 19, wherein the reagent capable of transforming hydrophilic amino acid residues into hydrophobic residues is selected from the group comprising alkylating, acylating, arylating, diazo, peptide-bond forming, Schiff's base forming compounds, or compounds capable of introducing a hydrophobic moiety.

22. The method according to claim 19, wherein the active water-soluble protein is selected from the group comprising enzymes, hormones, receptors, antigens, antibodies, allergens, immune-reactive proteins, affinity partners and derivatives, fragments and functionally equivalents thereof.

23. The method according to claim 22, wherein the active water-soluble protein is selected from the group comprising muscle aldolase, trypsin, amylase, lipase, collagenase or elastase.

24. The method according to claim 19 wherein the hydrophobic solid support is natural or synthetic polymers, carbon materials, silicon, metal, resins, all optionally derivatized to make them hydrophobic.

25. The method according to claim 24, wherein the hydrophobic solid support is in the form of wafers, slabs, laminas, sheets, tubes, fibers, particles, granulates, powders.

26. A device obtainable by the method according to claim 19.

27. The device according to claim 26 in the form of a cartridge packed in a laboratory tip or in the form of a filter or in the form of a microarray support.
